(19) United States
(12) Patent Application Publication
(10) Pub. No.: US 2022/0218048 A1
(43) Pub. Date: Jul. 14, 2022

(54) CLOTHING-TYPE WEARABLE FABRIC CAPABLE OF ADJUSTING TEMPERATURE THEREOF

(71) Applicants: Avary Holding (Shenzhen) Co., Limited., Shenzhen (CN); QING DING PRECISION ELECTRONICS (HUAIAN) CO.,LTD, Huai an (CN); GARUDA TECHNOLOGY CO., LTD., New Taipei (TW)

(72) Inventor: MAO-FENG HSU, Taoyuan (TW)

(21) Appl. No.: 17/167,887

(22) Filed: Feb. 4, 2021

(30) Foreign Application Priority Data
Jan. 12, 2021 (CN) 202110037653.7

(51) Int. Cl. A41D 13/005; A41D 1/00

(52) U.S. Cl. CPC A41D 13/0051; A41D 2400/12; A41D 1/005

(57) ABSTRACT

A clothing-type fabric capable of heating itself and cooling itself for the bodily comfort of a wearer includes a fabric body, at least two first electrodes, a second electrode, and a processor. The fabric body includes an inner surface and an outer surface opposite to the inner surface. The at least two first electrodes are disposed above the inner surface and the outer surface. The first electrode disposed above the inner surface can sense a body temperature of a wearer (first temperature value). The first electrode disposed above the outer surface can sense temperature of ambient environment (second temperature value). The second electrode is disposed above the inner surface, and can release heat and absorb heat. The processor can receive the first and the second temperature values, and control the second electrode to release or absorb heat by reference to the first temperature value and the second temperature value.

### FIELD

The subject matter herein generally relates to temperature regulation, and more particularly, to a clothing-type wearable fabric capable of adjusting temperature.

### BACKGROUND

Artificial skin with sensors, robotic arms, and certain clothing, may include wearable fabrics which have functions that are powered. Existing wearable fabric does not adjust its temperature, which fails to meet actual needs of users. Improvement in the art is desired.

### BRIEF DESCRIPTION OF THE DRAWINGS

Implementations of the present technology will now be described, by way of embodiment, with reference to the attached figures.

FIG. 1 is a diagrammatic view of an embodiment of a clothing-type wearable fabric capable of adjusting temperature, according to the present disclosure.

FIG. 2 is a diagrammatic view of two temperature adjusting modules of the wearable fabric of FIG. 1.

FIG. 3 is a cross-sectional view along of FIG. 1.

FIG. 4 is a diagrammatic view of a signal transmission line of the wearable fabric of FIG. 1.

FIG. 5 is a diagrammatic view of the temperature adjusting module of FIG. 2 before and after being stretched.

FIG. 6 is a cross-sectional view of an embodiment of a stretchable circuit of the signal transmission line of FIG. 4.

FIG. 7 is a cross-sectional view of another embodiment of a stretchable circuit of the signal transmission line of FIG. 4.

FIG. 8 is a cross-sectional view of yet another embodiment of a stretchable circuit of the signal transmission line of FIG. 4.

FIG. 9 is a cross-sectional view of yet another embodiment of a stretchable circuit of the signal transmission line of FIG. 4.

FIG. 10 is a cross-sectional view of yet another embodiment of a stretchable circuit of the signal transmission line of FIG. 4.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

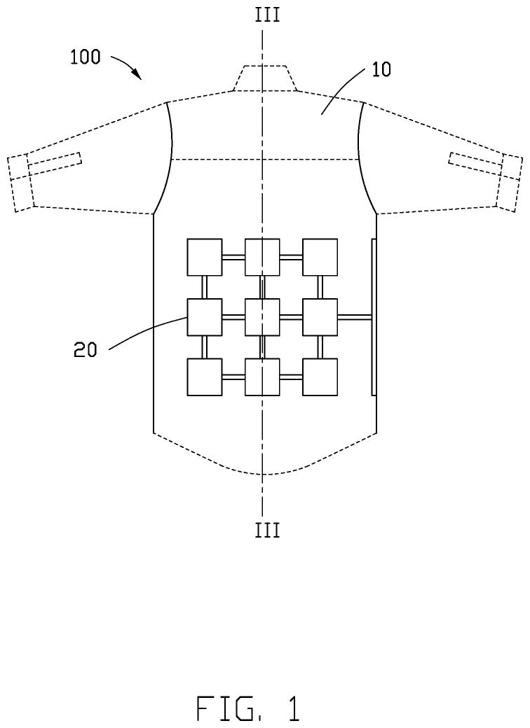

FIG. 1 illustrates an embodiment of a clothing-type wearable fabric 100 capable of adjusting its own temperature. The wearable fabric 100 can be worn around a human body. The wearable fabric 100 includes a fabric body 10 and a temperature adjusting module 20 disposed on the fabric body 10. The fabric body 10 can be of a jacket or pants. The fabric body 10 can insulate heat from the ambient environment.

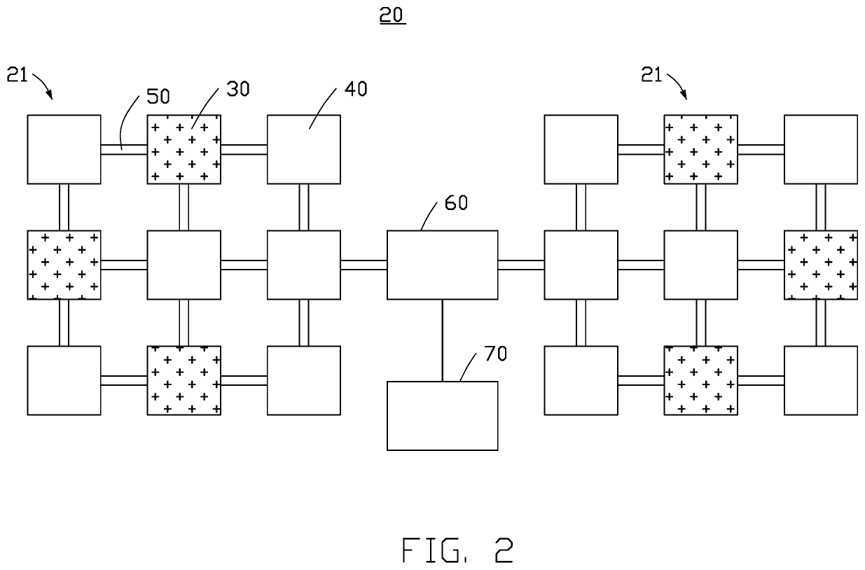

Referring to FIG. 2, the temperature adjusting module 20 includes at least one temperature adjusting array 21 (FIG. 2 shows that there are two temperature adjusting arrays 21). Each temperature adjusting array 21 includes at least two first electrodes 30, at least one second electrode 40, and a plurality of signal transmission lines 50. The first electrodes 30 and the second electrodes 40 are cooperatively arranged in an array. The signal transmission lines 50 cross-link the first electrodes 30 and the second electrode(s) 40 together to form grids, thereby improving a structure stability of the temperature adjusting array 21. That is, two adjacent first electrodes 30 are connected to each other through the signal transmission line 50. When there are two second electrodes 40, two adjacent second electrodes 40 are connected to each other through the signal transmission line 50. The first electrode 30 and the adjacent second electrode 40 are also connected to each other through the signal transmission line 50.

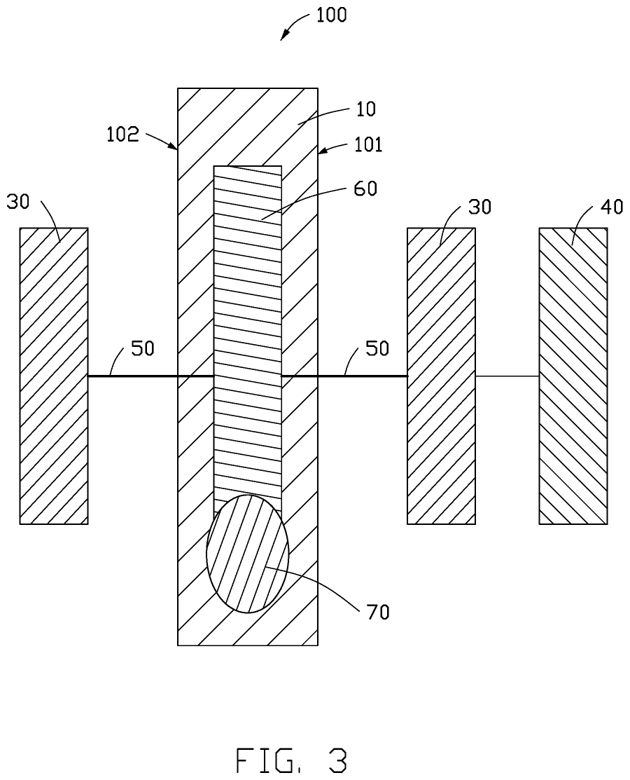

Referring to FIG. 3, the fabric body 10 includes an inner surface 101 and an outer surface 102 opposite to the inner surface 101. When the wearable fabric 100 is worn around the human body, the inner surface 101 faces the human body. At least one of the first electrodes 30 is disposed above the inner surface 101 of the fabric body 10. At least one of the first electrodes 30 is disposed above the outer surface 102 of the fabric body 10. The first electrode 30 disposed above the inner surface 101 can sense a temperature of the body of the wearer (first temperature value). The first electrode 30 disposed on the outer surface 102 can sense temperature of the ambient environment (second temperature value).

Each first electrode 30 is stretchable under an external force. The first electrode 30 includes a first stretchable substrate (not shown), a first stretchable pattern (not shown) disposed on the first stretchable substrate, and a first electrode layer (not shown) disposed on the first stretchable substrate and electrically connected to the first stretchable pattern. In at least one embodiment, the first stretchable pattern is made of silver paste. The first electrode layer can sense the first temperature value and the second temperature value.

The second electrode 40 is disposed above the inner surface 101 of the fabric body 10. The second electrode 40 can absorb heat and release heat when energized. When the first temperature value is low, the second electrode 40 can release heat to increase the temperature of the wearer. When the first temperature value is high, the second electrode 40 can absorb and dissipate heat to reduce the first temperature value.

Each second electrode 40 is stretchable under an external force. The second electrode 40 includes a second stretchable substrate (not shown), a second stretchable pattern (not shown) disposed on the second stretchable substrate, and a second electrode layer (not shown) disposed on the second stretchable substrate and electrically connected to the second stretchable pattern. In at least one embodiment, the second stretchable pattern is made of silver paste. The second electrode layer can also absorb heat and release heat.

The temperature adjusting module 20 further includes a processor 60. The processor 60 can be disposed inside the fabric body 10. The processor 60 is electrically connected to the first electrodes 30 and the second electrode(s) 40 through the signal transmission lines 50. The processor 60 can receive the first temperature value and the second temperature value from the first electrodes 30, and control the second electrode 40 to release or absorb heat by reference to the first temperature value and the second temperature value. As such, the wearable fabric 100 can adjust the body temperature of the wearer, thereby improving the comfort of the user.

In at least one embodiment, when the first temperature value and the second temperature value are equal to a first preset value and a second preset value respectively, the user should feel that his current temperature is comfortable. When at least one of the first temperature value and the second temperature value decreases, the processor 60 controls the second electrode 40 to release heat according to the amount of decrease of the first or second temperature value. When at least one of the first temperature value and the second temperature value increases, the processor 60 controls the second electrode 40 to absorb heat according to the amount of increase of the first or second temperature value.

In at least one embodiment, the second electrode 40 can further sense bioelectric signals of the human body. The bioelectric signals can include ECG signals, electromyography signals, a respiration rate, ocular vibration signals, and brain wave signals. The processor 60 can further receive the bioelectric signals, and determine the physiological state of the human body according to such bioelectric signals.

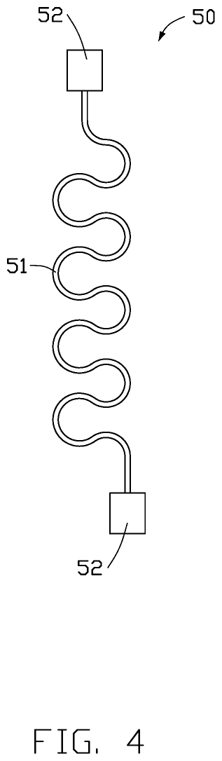

Referring to FIG. 4, each signal transmission line 50 is stretchable under an external force. The signal transmission lines 50 includes at least one stretchable circuit 51 and two terminals 52 disposed at opposite ends of the stretchable circuit 51. The terminal 52 is electrically connected to the processor 60, the first electrode 30, and the second electrode 40. The stretchable circuit 51 can have a horseshoe or zigzag shape.

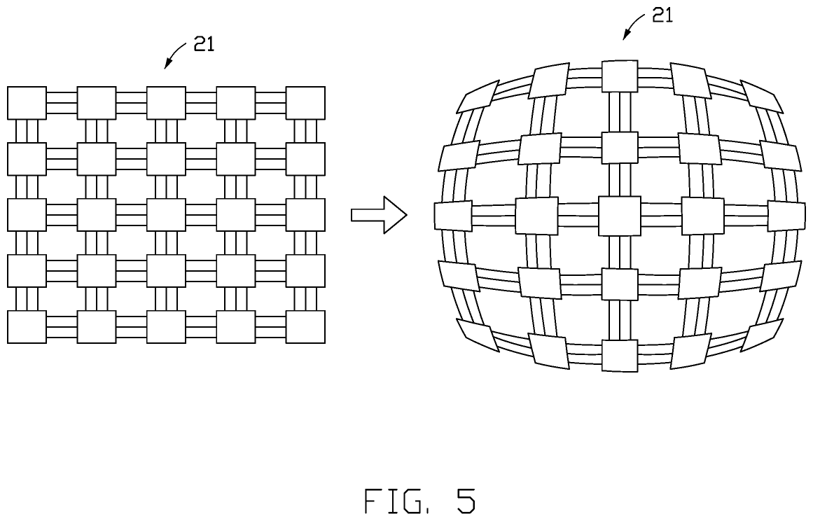

Referring to FIG. 5, since the first electrode 30, the second electrode 40, and the signal transmission line 50 can be stretched under an external force, the temperature adjusting array 21 can also be stretched. The temperature adjusting array 21 can further return to its initial state after the external force is removed.

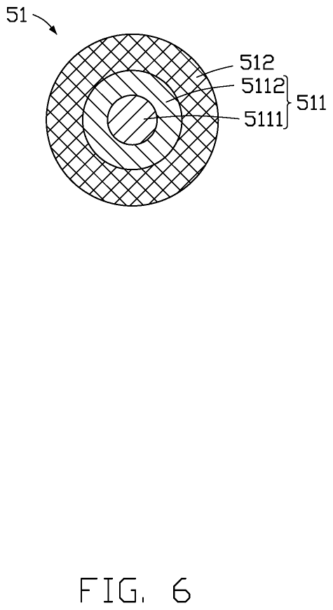

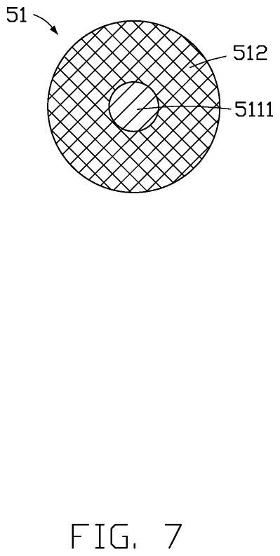

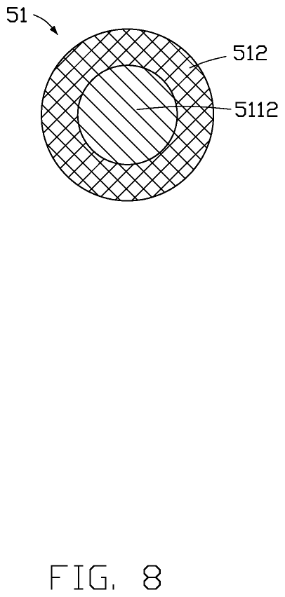

Referring to FIGS. 6 to 8, the stretchable circuit 51 includes a conductive core 511 and an insulating layer 512 around the conductive core 511. The conductive core 511 may be a single layer or multiple layers. For example, as shown in FIG. 6, the conductive core 511 includes a silver paste layer 5111 and a carbon paste layer 5112 around the silver paste layer 5111. As shown in FIGS. 7 and 8, the conductive core 511 only includes the silver paste layer 5111 or the carbon paste layer 5112. The insulating layer 512 can be made of a stretchable and resilient material, which returns to its original state when an external stretching force is removed, for example, the material can be thermoplastic polyurethane (TPU) or rubber.

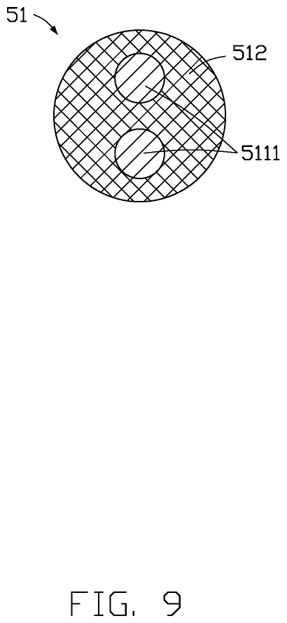

As shown in FIG. 9, the stretchable circuit 51 can also include two conductive cores 511 spaced apart from each other. The insulating layer 512 is also disposed between the two conductive cores 511.

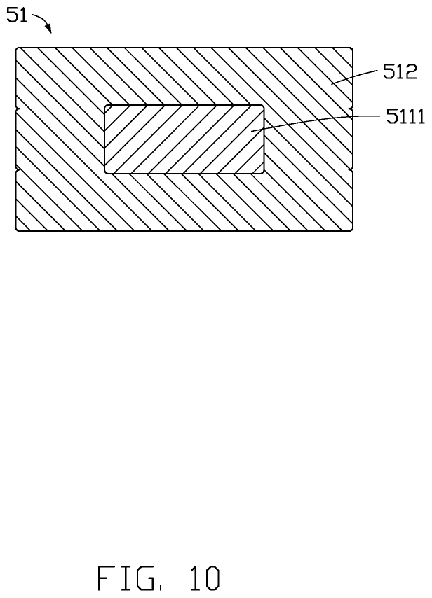

As shown in FIGS. 6-9, a cross-section of the stretchable circuit 51 is circular. Referring to FIG. 10, the cross-section of the stretchable circuit 51 can also be rectangular.

Referring to FIG. 3, the temperature adjusting module 20 further includes a power supply 70. The power source 70 is disposed inside the fabric body 10 and electrically connected to the processor 60. The power supply 70 can provide electrical energy to the first electrode 30, the signal transmission line 50, and the second electrode 40. The power source 70 may be a battery.

Even though information and advantages of the present embodiments have been set forth in the foregoing description, together with details of the structures and functions of the present embodiments, the disclosure is illustrative only. Changes may be made in detail, especially in matters of shape, size, and arrangement of parts within the principles of the present embodiments to the full extent indicated by the plain meaning of the terms in which the appended claims are expressed.

What is claimed is:

1. A clothing-type wearable fabric capable of adjusting temperature thereof, comprising:
a fabric body comprising an inner surface and an outer surface opposite to the inner surface;
at least two first electrodes, at least one of the first electrodes disposed above the inner surface and at least one of the first electrodes disposed above the outer surface, the first electrode disposed above the inner surface configured to sense a first temperature value of a human body, the first electrode disposed above the outer surface configured to sense a second temperature value of an ambient environment;
a second electrode disposed above the inner surface, and configured to release and absorb heat; and
a processor configured to receive the first temperature value and the second temperature value, and control the second electrode to release or absorb according to the first temperature value and the second temperature value.

2. The clothing-type wearable fabric of claim 1, wherein the second electrode is further configured to sense bioelectric signals of the human body, and the processor is further configured to determine a physiological state of the human body according to the bioelectric signals.

3. The clothing-type wearable fabric of claim 1, wherein the wearable fabric comprises at least two second electrodes and a plurality of signal transmission lines, the first electrodes and the second electrodes are cooperatively arranged in an array, and the plurality of signal transmission lines cross-links the first electrodes and the second electrodes together.

4. The clothing-type wearable fabric of claim 3, wherein two adjacent first electrodes are connected to each other through one of the plurality of signal transmission lines, two adjacent second electrodes are connected to each other through one of the plurality of signal transmission lines, and the first electrode and an adjacent second electrode are connected to each other through one of the plurality of signal transmission lines.

5. The clothing-type wearable fabric of claim 3, wherein the first electrode, the second electrode, and the plurality of signal transmission lines are stretchable under an external force.

6. The clothing-type wearable fabric of claim 3, wherein each of the plurality of signal transmission lines comprises a stretchable circuit and two terminals arranged at opposite ends of each stretchable circuit, and each terminal is electrically connected to the processor, the first electrode, or the second electrode.

7. The clothing-type wearable fabric of claim 6, wherein stretchable circuit has a horseshoe shape or a zigzag shape.

8. The clothing-type wearable fabric of claim 6, wherein the stretchable circuit comprises at least one conductive core and an insulating layer around the conductive core.

9. The clothing-type wearable fabric of claim 8, wherein the conductive core comprises a silver paste layer and a carbon paste layer around the silver paste layer.

10. The clothing-type wearable fabric of claim 8, wherein the conductive core is a silver paste layer or a carbon paste layer.

11. The clothing-type wearable fabric of claim 8, wherein the stretchable circuit comprises two conductive cores spaced apart from each other, and the insulating layer is also disposed between the two conductive cores.

12. The clothing-type wearable fabric of claim 1, further comprising a power supply electrically connected to the processor.

13. The clothing-type wearable fabric of claim 12, wherein the processor and the power supply are disposed inside the fabric body.
